(19) United States
(12) Patent Application Publication
(10) Pub. No.: US 2006/0263328 A1
(43) Pub. Date: Nov. 23, 2006

(54) Hydrophilic polymers with pendant functional groups and method thereof

(76) Inventors: Sang Van, San Diego, CA (US); Lei Yu, Carlsbad, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/134,820

(22) Filed: May 19, 2005

(51) Int. Cl. A61K 31/787; C08G 63/91; C08G 63/48

(52) U.S. Cl. 424/78.27; 525/54.2; 525/437

(57) ABSTRACT

A polymer comprising a recurring unit of the formula (I) is described.

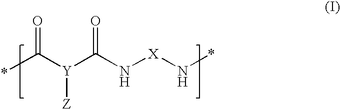

In the formula (I), X is selected from the group consisting of —(CH₂CH₂O)ₘ—CH₂CH₂— and —CH₂CH₂CH₂O—(CH₂CH₂O)ₘ—CH₂CH₂CH₂—, wherein m is an integer in the range of 1 to 100; Y is selected from the group consisting of —C*HCH₂—, —C*HCH₂CH₂—, —C*H—NHC(═O)—CH₂CH₂—, —C*H—NHC(═O)—CH₂CH₂CH₂—, —CH₂CH₂N*CH₂CH₂—, —CH₂CH₂CH₂N*CH₂CH₂CH₂—, C₂ to C₂₀ alkyl, and C₆ to C₂₀ aryl, wherein C* and N* represent atoms to which Z is bonded; and Z is selected from the group consisting of —NHR¹, —NH—C(═O)—(CH₂)ₙC(═O)NR¹R¹, —NH—C(═O)—(CH₂)ₙC(═O)OR¹, —(CH₂)ₙC(═O)NR¹R², —(CH₂)ₙC(═O)OR¹, —(CH₂)ₙC(═O)SR¹, and —(CH₂)ₙNR¹R², wherein n is an integer in the range of 1 to 3, wherein R¹ and R² are each independently selected from the group consisting of hydrogen, C₁ to C₂₀ alkyl, C₆ to C₂₀ aryl, anticancer drugs, peptides, antibody fragment, lactose, galactose, mannose, transferrin, magnetic resonance imaging agents, succinimyl, and alkali metal.

### BACKGROUND OF THE INVENTION

1. Field of the Invention

This invention relates generally to novel biocompatible hydrophilic polymers with pendant functional groups and methods for making them. These polymers are useful for a variety of drug, biomolecule and imaging agent delivery applications.

2. Description of the Related Art

A variety of systems have been used for the delivery of drugs, biomolecules, and imaging agents. For example, such systems include capsules, liposomes, microparticles, nanoparticles, and polymers. Polymers are often classified as being either biodegradable or nonbiodegradable.

A variety of polyester-based biodegradable systems have been characterized and studied. Polylactic acid (PLA), polyglycolic acid (PGA) and their copolymers polylactic-co-glycolic acid (PLGA) are some of the most well-characterized biomaterials with regard to design and performance for drug-delivery applications. See Uhrich, K. E.; Cannizzaro, S. M.; Langer, R. S. and Shakeshelf, K. M. “Polymeric Systems for Controlled Drug Release.” Chem. Rev. 1999, 99, 3181-3198 and Panyam J, Labhasetwar V. “Biodegradable nanoparticles for drug and gene delivery to cells and tissue.” Adv Drug Deliv Rev. 2003, 55, 329-47. Biodegradable systems based on polyorthoesters have also been investigated. See Heller, J.; Barr, J.; Ng, S. Y.; Abdellauoi, K. S. and Gumy, R. “Poly(ortho esters): synthesis, characterization, properties and uses.” Adv. Drug Del. Rev. 2002, 54, 1015-1039. Polyanhydride systems have also been investigated. Such polyanhydrides are typically biocompatible and may degrade in vivo into relatively non-toxic compounds that are eliminated from the body as metabolites. See Kumar, N.; Langer, R. S. and Domb, A. J. “Polyanhydrides: an overview.” Adv. Drug Del. Rev. 2002, 54, 889-91.

Amino acid-based polymers have also been considered as a potential source of new biomaterials. Poly-amino acids having good biocompatibility have been investigated to deliver low molecular-weight compounds. A relatively small number of polyglutamic acid and copolymers have been identified as candidate materials for drug delivery. See Bourke, S. L. and Kohn, J. “Polymers derived from the amino acid L-tyrosine: polycarbonates, polyarylates and copolymers with poly(ethylene glycol).” Adv. Drug Del. Rev., 2003, 55, 447-466.

Administered hydrophobic anticancer drugs and therapeutic proteins and polypeptides often suffer from poor bio-availability. Such poor bio-availability may be due to incompatibility of bi-phasic solutions of hydrophobic drugs and aqueous solutions and/or rapid removal of these molecules from blood circulation by enzymatic degradation. One technique for increasing the efficacy of administered proteins and other small molecule agents entails conjugating the administered agent with a polymer, such as a polyethylene glycol (“PEG”) molecule, that can provide protection from enzymatic degradation in vivo. Such “PEGylation” often improves the circulation time and, hence, big-availability of an administered agent.

PEG has shortcomings in certain respects, however. For example, because PEG is a linear polymer, the steric protection afforded by PEG is limited, as compared to branched polymers. Another major shortcoming of PEG is that it is only amenable to derivatization at its two terminals. This limits the number of other functional molecules (e.g. those helpful for protein or drug delivery to specific tissues) that can be conjugated to a PEG.

### SUMMARY OF THE INVENTION

The inventors have used relatively small ethylene glycol and amino acid derivatives to make novel hydrophilic polymers, e.g., as schematically illustrated in FIG. 2.

In some embodiments, the starting materials used to make the polymers include ethylene glycols (FDA-approved biomaterials) and amino acids (natural products), which are typically biocompatible and degradable. Preferred polymers may be used for various bio-delivery applications.

Embodiments of the invention are directed to polymers comprising a recurring unit of the formula (I):

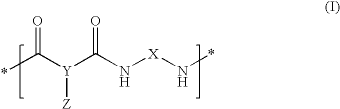

wherein: X is selected from the group consisting of —(CH₂CH₂O)ₘ—CH₂CH₂— and —CH₂CH₂CH₂O—(CH₂CH₂O)ₘ—CH₂CH₂CH₂—, wherein m is an integer in the range of 1 to 100; Y is selected from the group consisting of —C*HCH₂—, —C*HCH₂CH₂—, —C*H—NHC(═O)—CH₂CH₂—, —C*H—NHC(═O)—CH₂CH₂CH₂—, —CH₂CH₂N*CH₂CH₂—, —CH₂CH₂CH₂N*CH₂CH₂CH₂—, C₂ to C₂₀ alkyl, and C₆ to C₂₀ aryl, wherein C* and N* represent atoms to which Z is bonded; and Z is selected from the group consisting of —NHR¹, —NH—C(═O)—(CH₂)ₙC(═O)NR¹R², —NH—C(═O)—(CH₂)ₙC(═O)OR¹, —(CH₂)ₙC(═O)NR¹R², —(CH₂)ₙC(═O)OR¹, —(CH₂)ₙC(═O)SR¹, and —(CH₂)ₙNR¹R², wherein n is an integer in the range of 1 to 3, wherein R¹ and R² are each independently selected from the group consisting of hydrogen, C₁ to C₂₀ alkyl, C₆ to C₂₀ aryl, anticancer drugs, peptides, antibody fragment, lactose, galactose, mannose, transferrin, magnetic resonance imaging agents, succinimyl, and alkali metal.

In preferred embodiments, the formula (I) represents the following formula (II):

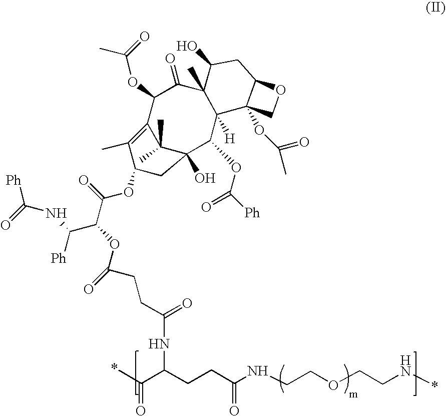

In preferred embodiments, the formula (I) represents the following formula (III):

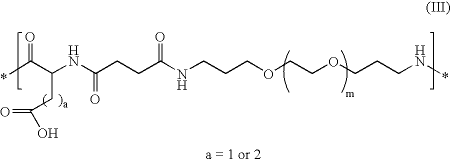

In preferred embodiments, the formula (I) represents the following formula (IV):

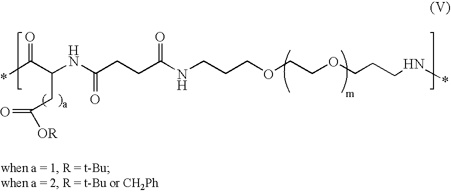

In preferred embodiments, the formula (I) represents the following formula (V):

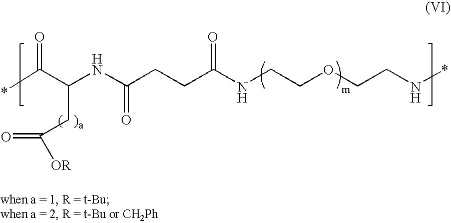

In preferred embodiments, the formula (I) represents the following formula (VI):

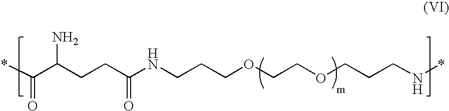

In preferred embodiments, the formula (I) represents the following formula (VII):

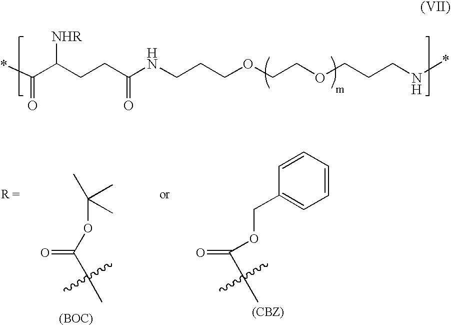

In preferred embodiments, the formula (I) represents the following formula (VIII):

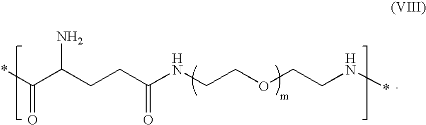

In preferred embodiments, the formula (I) represents the following formula (IX):

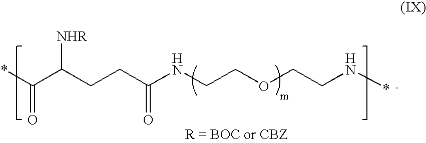

Embodiments of the invention are directed to compositions comprising the formula (X):

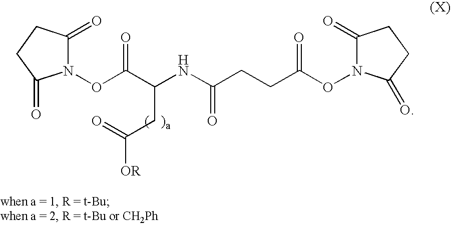

Embodiments of the invention are directed to a method of producing polymers that include a recurring unit of the formula (II) which comprises reacting a polymer comprising a recurring unit of the formula (VIII) with a Pac-NHS.

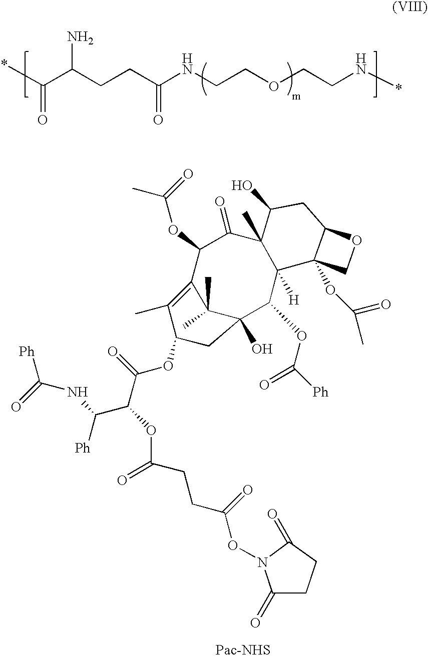

Embodiments of the invention are directed to a method of producing polymers that include a recurring unit of the formula (III) which comprises reacting a polymer comprising a recurring unit of the formula (IV) with trifluoacetic acid (TFA) or catalytic palladium/carbon hydrogenation.

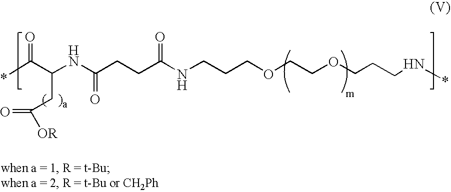

Embodiments of the invention are directed to a method of producing polymers that include a recurring unit of the formula (IV) which comprises reacting a compound of the formula (X) with a compound of the formula (XII).

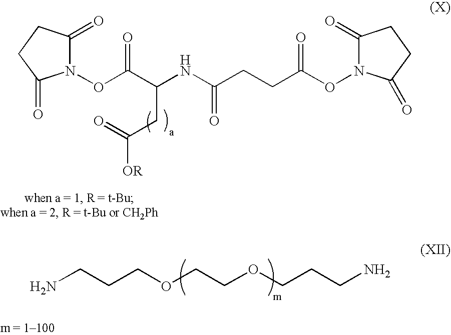

Embodiments of the invention are directed to a method of producing polymers that include a recurring unit of the formula (V) which comprises reacting a compound of the formula (X) with a compound of the formula (XIII).

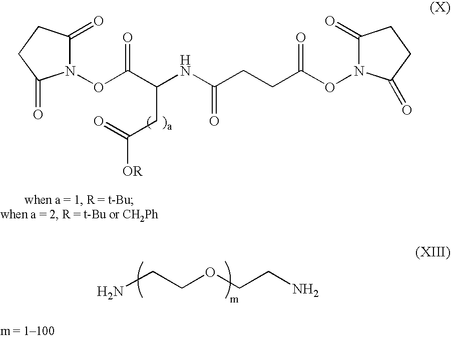

Embodiments of the invention are directed to a method of producing polymers that include a recurring unit of the formula (VI) which comprises reacting a polymer comprising a recurring unit of the formula (VII) with TFA or catalytic palladium/carbon hydrogenation.

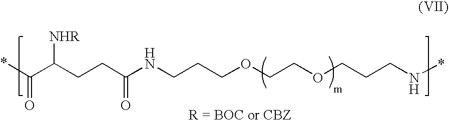

Embodiments of the invention are directed to a method of producing polymers that include a recurring unit of the formula (VII) which comprises reacting a compound of the formula (XI) with a compound of the formula (XII).

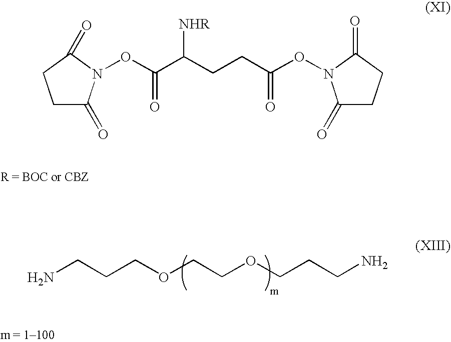

Embodiments of the invention are directed to a method of producing polymers that include a recurring unit of the formula (VIII) which comprises reacting a polymer comprising a recurring unit of the formula (IX) with TFA or catalytic palladium/carbon hydrogenation.

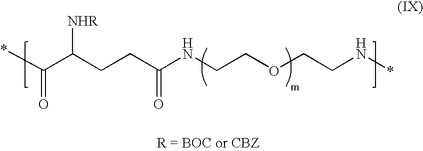

Embodiments of the invention are directed to a method of producing polymers that include a recurring unit of the formula (IX) which comprises reacting a compound of the formula (XI) with a compound of the formula (XIII).

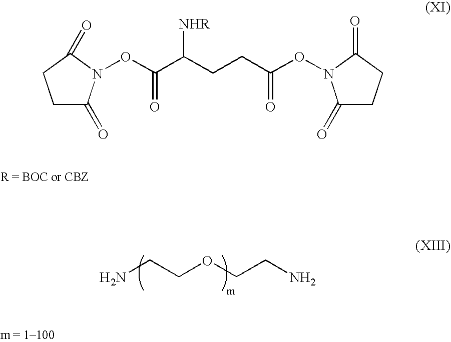

Embodiments of the invention are directed to a method of producing compositions comprising the formula (X) which comprises reacting L-glutamic acid γ-benzyl ester, L-aspartic acid β-t-butyl ester, or L-glutamic acid γ-t-butyl ester with succinimide anhydride, followed by 1-(3-dimethylaminopropyl)-3-ethylcardiimide hydrochloride (EDC), N-hydroxysuccinimide (NHS) coupling.

Detailed descriptions of hydrophilic polymers for drug delivery are described in the following references, which are hereby incorporated by reference: U.S. Pat. No. 6,653,427; U.S. Pat. No. 6,706,836; U.S. Pat. No. 6,743,880; U.S. Pat. No. 5,962,620; U.S. Pat. No. 5,993,972; U.S. 2002/155158; U.S. 2004/185103; U.S. Pat. No. 6,706,289; U.S. Pat. No. 6,652,886; U.S. 2004/0228831; U.S. 2004/0170595; U.S. 2004/0161403; U.S. 2003/0220447; U.S. 2003/0147958; U.S. 2003/0018002; U.S. Pat. No. 6,515,100; U.S. Pat. No. 6,541,015 and WO2003/066069.

Further aspects, features and advantages of this invention will become apparent from the detailed description of the preferred embodiments which follow.

### BRIEF DESCRIPTION OF THE DRAWINGS

These and other feature of this invention will now be described with reference to the drawings of preferred embodiments which are intended to illustrate and not to limit the invention.

FIG. 1 shows conventional use of poly(ethylene glycol) (PEG) as a block co-polymer.

FIG. 2 shows use of ethylene glycol as a small spacer unit in a larger polymer according to an embodiment.

FIG. 3 shows a reaction scheme for making a polymer according to an embodiment.

FIG. 4 shows a reaction scheme for making a polymer according to another embodiment.

FIG. 5 shows a reaction scheme for the preparation of compounds 13-17.

FIG. 6 shows a reaction scheme for making a polymer according to another embodiment.

FIG. 7 shows a reaction scheme for making a polymer according to another embodiment.

FIG. 8 shows a reaction scheme for making a polymer according to another embodiment.

FIG. 9 shows a reaction scheme for making a polymer according to another embodiment.

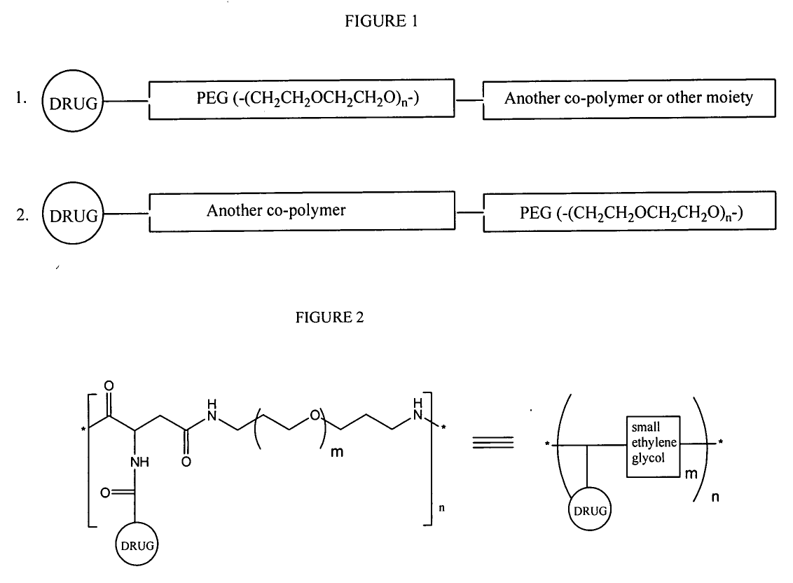

### DETAILED DESCRIPTION OF THE PREFERRED EMBODIMENTS

These and other features of this invention will now be described with reference to the drawings of preferred embodiments which are intended to illustrate and not to limit the invention.

An embodiment provides a polymer comprising a recurring unit of the formula (I):

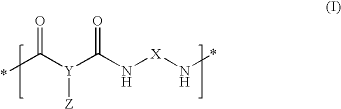

wherein: X is selected from the group consisting of —(CH₂CH₂O)ₘ—CH₂CH₂— and —CH₂CH₂CH₂O—(CH₂CH₂O)ₘ—CH₂CH₂CH₂—, wherein m is an integer in the range of 1 to 100; Y is selected from the group consisting of —C*HCH₂—, —C*HCH₂CH₂—, —C*H—NHC(═O)—CH₂CH₂—, —C*H—NHC(═O)—CH₂CH₂CH₂—, —CH₂CH₂N*CH₂CH₂—, —CH₂CH₂CH₂N*CH₂CH₂CH₂—, C₂ to C₂₀ alkyl, and C₆ to C₂₀ aryl, wherein C* and N* represent atoms to which Z is bonded; and Z is selected from the group consisting of —NHR¹, —NH—C(═O)—(CH₂)ₙC(═O)NR¹R², —NH—C(═O)—(CH₂)ₙC(═O)OR¹, —(CH₂)ₙC(═O)NR¹R², —(CH₂)ₙC(═O)OR¹, —(CH₂)ₙC(═O)SR¹, and —(CH₂)ₙNR¹R², wherein n is an integer in the range of 1 to 3, wherein R¹ and R² are each independently selected from the group consisting of hydrogen, C₁ to C₂₀ alkyl, C₆ to C₂₀ aryl, anticancer drugs, peptides, antibody fragment, lactose, galactose, mannose, transferrin, magnetic resonance imaging agents, succinimyl, and alkali metal.

Examples of polymers that comprises recurring units of the formula (I) include polymers that comprise recurring units of the formula (II), polymers that comprise recurring units of the formula (III), polymers that comprise recurring units of the formula (IV), polymers that comprise recurring units of the formula (V), polymers that comprise recurring units of the formula (VI), polymers that comprise recurring units of the formula (VII), polymers that comprise recurring units of the formula (VIII), and polymers that comprise recurring units of the formula (IX):

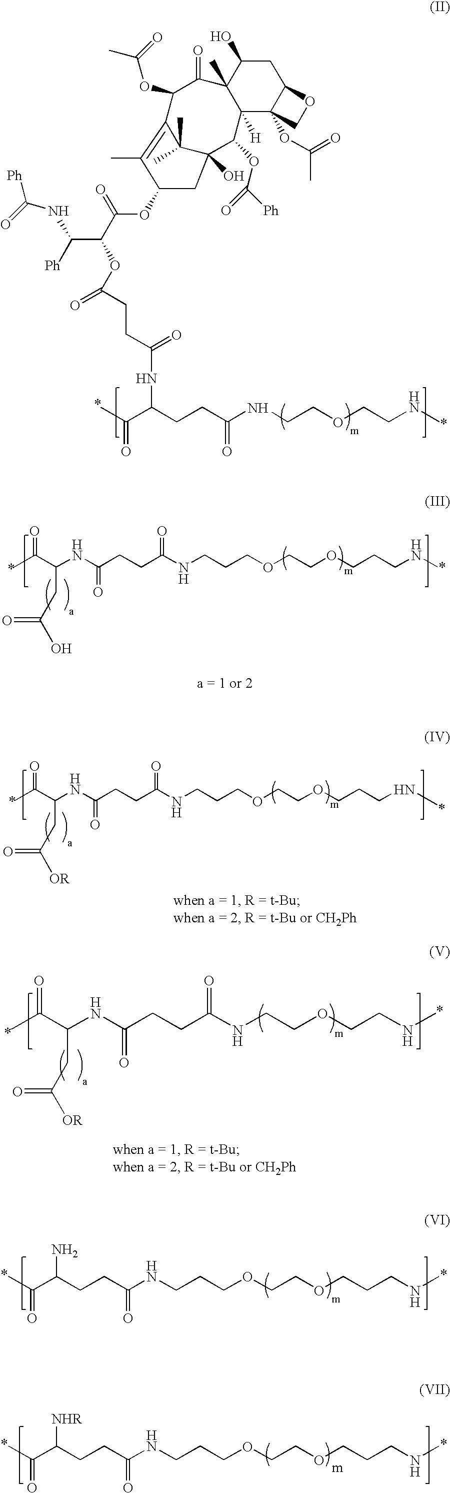

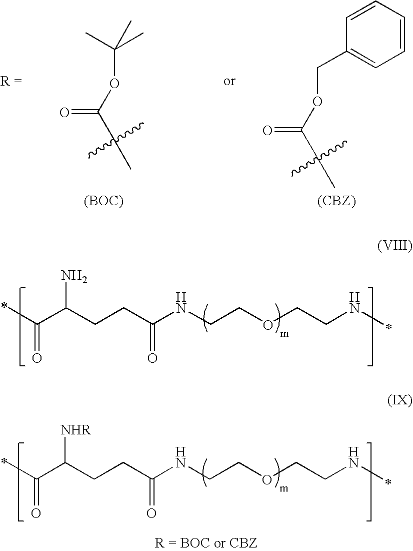

Polymers that comprise recurring units of the formula (II) may be prepared by a process that comprises reacting a polymer comprising a recurring unit of the formula (VIII) with Pac-NHS.

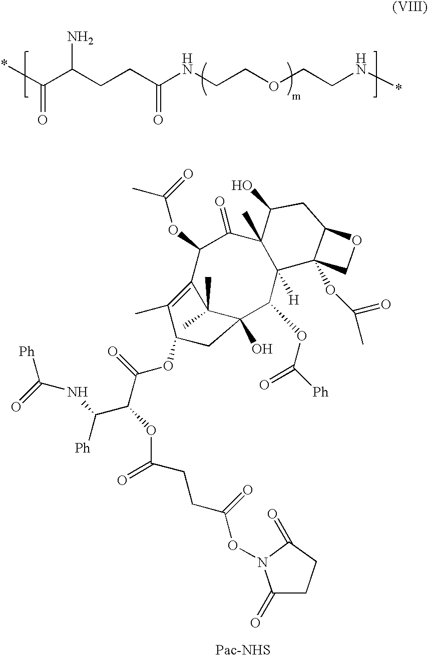

Polymers that comprise recurring units of the formula (III) may be prepared by a process that comprises reacting a polymer comprising a recurring unit of the formula (IV) with trifluoacetic acid (TFA) or catalytic palladium/carbon hydrogenation.

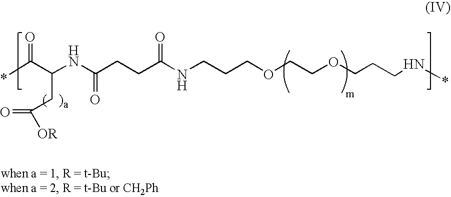

Polymers that comprise recurring units of the formula (IV) may be prepared by a process that comprises reacting a compound of the formula (X) with a compound of the formula (XII).

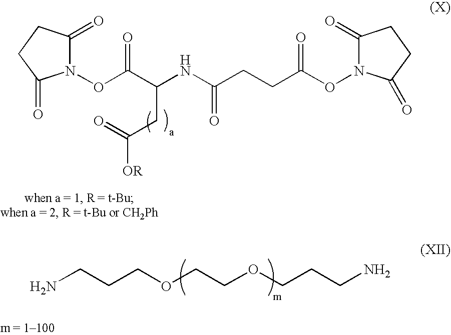

Polymers that comprise recurring units of the formula (V) may be prepared by a process that comprises reacting a compound of the formula (X) with a compound of the formula (XIII).

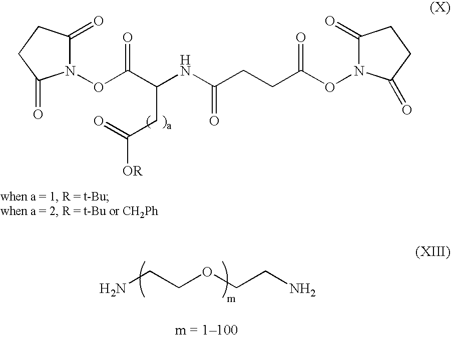

Polymers that comprise recurring units of the formula (VI) may be prepared by a process that comprises reacting a polymer comprising a recurring unit of the formula (VII) with TFA or catalytic palladium/carbon hydrogenation.

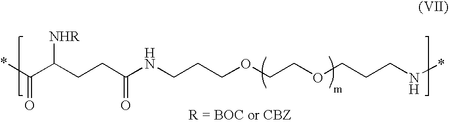

Polymers that comprise recurring units of the formula (VII) may be prepared by a process that comprises reacting a compound of the formula (XI) with a compound of the formula (XII).

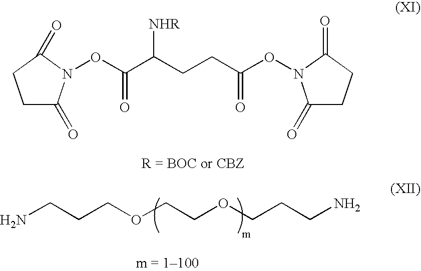

Polymers that comprise recurring units of the formula (VIII) may be prepared by a process that comprises reacting a polymer comprising a recurring unit of the formula (IX) with TFA or catalytic palladium/carbon hydrogenation.

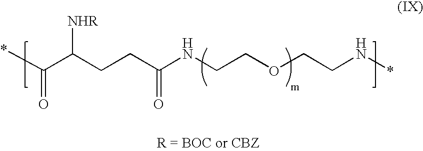

Polymers that comprise recurring units of the formula (IX) may be prepared by a process that comprises reacting a compound of the formula (XI) with a compound of the formula (XIII).

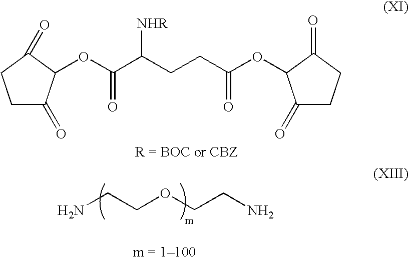

An embodiment provides a composition comprising a compound represented by the formula (X):

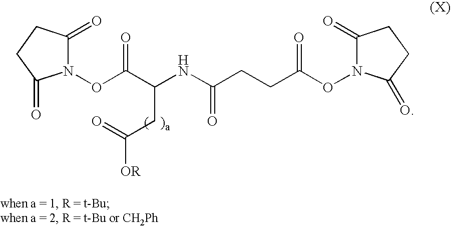

Compositions that comprise a compound of the formula (X) may be prepared by a process that comprises reacting L-glutamic acid γ-benzyl ester, L-aspartic acid β-t-butyl ester, or L-glutamic acid γ-t-butyl ester with succinimide anhydride, followed by 1-(3-Dimethylaminopropyl)-3-ethylcardiimide hydrochloride (EDC), N-hydroxysuccinimide (NHS) coupling.

### EXAMPLES

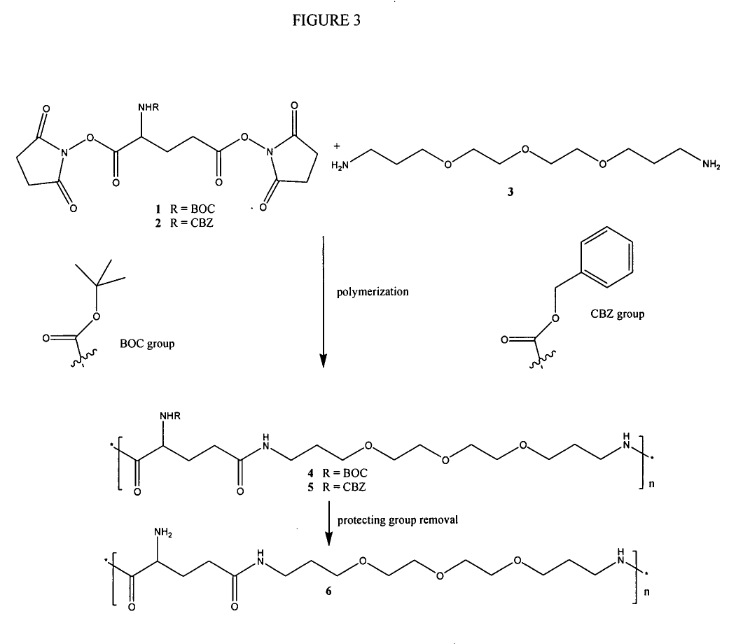

The chemical structures of compounds 1-26 are shown in FIGS. 3-9. Synthesis of compound 1 and compound 2 was carried out by the well-known method of EDC coupling using NHS from N-α-t-boc-L-glutamic acid and N-α-CBZ-L-glutamic acid (EMD Biosciences Inc.), respectively. Compound 3 was purchased from TCI Chemicals, Inc. L-glutamic acid γ-benzyl ester, L-aspartic acid β-t-butyl ester and L-glutamic acid γ-t-butyl ester were purchased from EMD Biosciences, Inc. Pac-NHS was synthesized according to the method described in Thierry et al. J Am Chem Soc. 2005 16;127(6):1626-7. Compound 23 was purchased from Nektar Therapeutics, Inc. Other chemicals, reagents and solvents were purchased from Aldrich Chemicals Company. Molecular weights are weight average and were determined by aqueous gel permeation chromatography (GPC) using polyethylene glycol standards. Chemical structures were confirmed by ¹H and ¹³C NMR spectra measured at room temperature on a 400 MHz (100 MHz for ¹³C) instrument in CDCl₃, D₂O, or DMSO-d₆.

### Example 1

A polymer 6 was produced according to the reaction scheme illustrated in FIG. 3 as follows: A solution of compound 3 (0.51 g, 2.3 mmol) in dichloromethane (DCM, 10 mL) was added to a solution of compound 1 (1.48 g, 2.3 mmol). The reaction was stirred for 15 hours. Water (50 mL) was added, and organic phases were extracted (50 mL×2), combined, dried with anhydrous sodium sulfate, filtered, and concentrated by rotary evaporation to yield a polymer 4 (0.80 g, 1.6 mmol, 0.71% yield). The polymer 4 was treated with 95% trifluoacetic acid (TFA) in DCM for 2 hours to yield the polymer 6 (0.30 g, 0.9 mmol, 56%) after dialysis with semi-permeable cellulose (cut-off molecular weight 3,500 daltons) and lyophilization.

### Example 2

A polymer 5 was produced according to the reaction scheme illustrated in FIG. 3 in a manner similar to that described in Example 1, except that compound 2 was used in place of compound 1 as illustrated in FIG. 3. Synthesis of polymer 6 from polymer 5 was carried out using a catalytic palladium/carbon hydrogenation instead of using TFA.

### Example 3

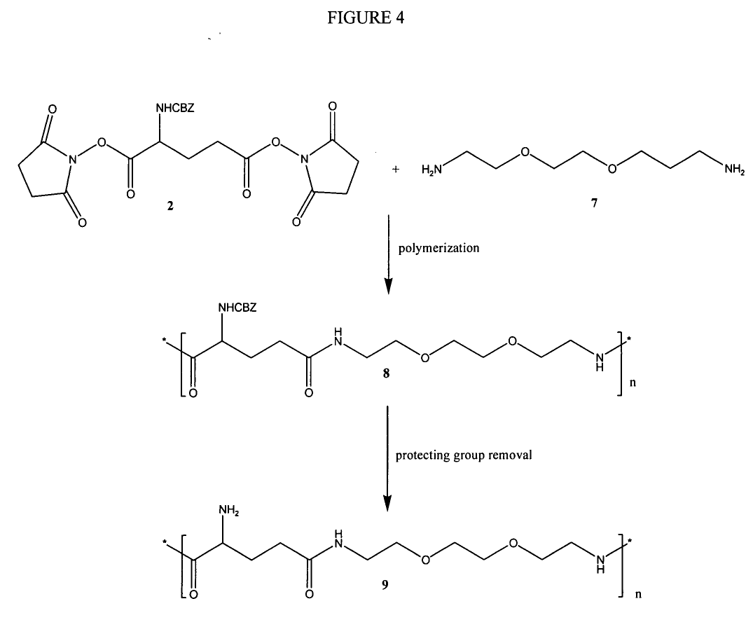

A polymer 9 was produced according to the reaction scheme illustrated in FIG. 4 in a manner similar to that described in Example 2, except that compound 7 was used in place of compound 3.

### Example 4

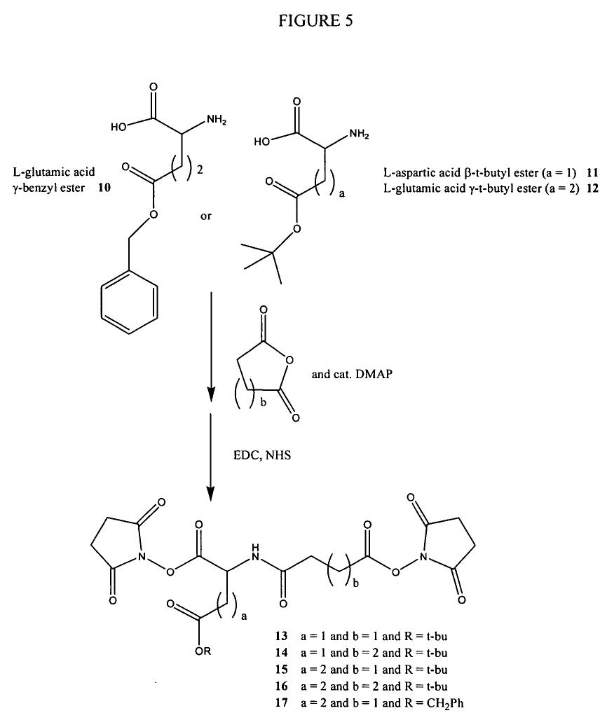

A compound 16 was produced according to the reaction scheme illustrated in FIG. 5 as follows: L-glutamic acid γ-t-butyl ester 12 (10.0 g, 49.2 mmol) and succinic anhydride (6.40 g, 64.0 mmol) and catalytic 4-dimethylaminopyridine (100 mg) were stirred in DMF (200 mL) for 15 hours. 1-(3-Dimethylaminopropyl)-3-ethylcardiimide hydrochloride (EDC, 25 g, 13.0 mmol) was added and continued to stir for 5 minutes, then N-hydroxysuccinimide (NHS, 15.5 g, 13.0 mmol) was added and continued to stir for 2 hours (coupling). The solvent was removed by rotary evaporation. The residue was redissolved in DCM (200 mL) and extracted two times from water. Organic phases were combined, dried with anhydrous sodium sulfate, filtered, and concentrated by rotary evaporation. The compound 16 (13.5 g, 27.2 mmol, 55%) was obtained after silica gel column purification with ethylacetate as an eluent (TLC, R_{f}=0.5).

### Examples 5-8

Compounds 13-15 and 17 were produced according to the reaction scheme illustrated in FIG. 5 in a matter similar to that described in Example 4, except that various combinations of compounds 10, 11 or 12 and succinic or glutaric anhydride were used.

### Example 9-10

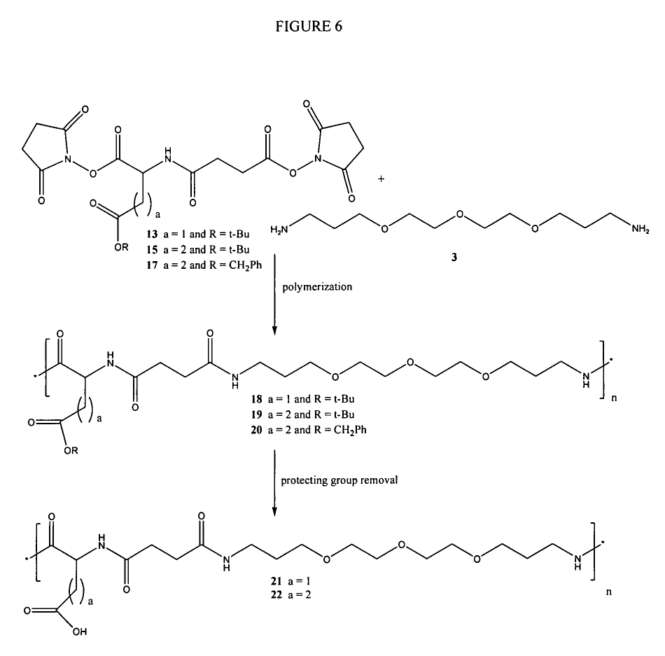

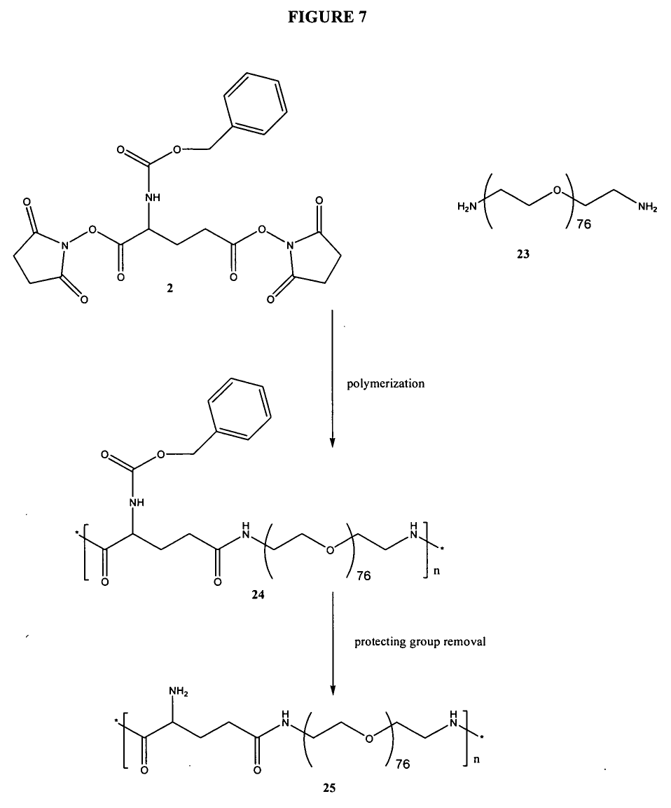

Polymers 21 and 22 were produced according to the reaction scheme illustrated in FIG. 6 in a manner similar to that described in Example 1, except that compound 13, 15 or 17 was used in place of compound 1. Synthesis of compounds 18-20 was similar to synthesis of compound 4, and synthesis of compounds 21 and 22 from compounds 18 and 19, respectively, was similar to synthesis of compound 6 from compound 4. Synthesis of compound 22 from compound 20 was similar to synthesis of compound 6 from compound 5 which was described in Example 2.

### Example 11

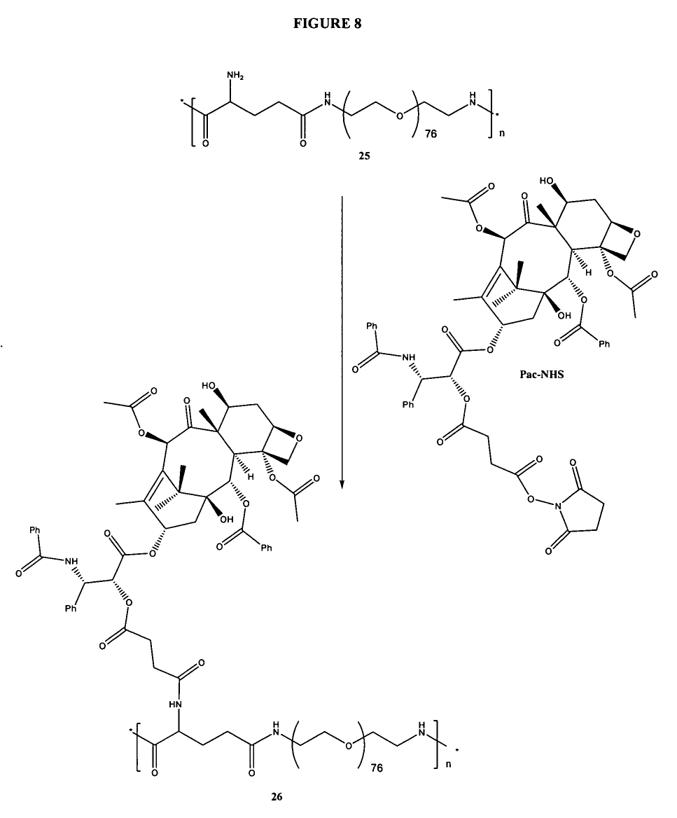

A polymer 25 was produced according to the reaction scheme illustrated in FIG. 7 in a manner similar to that described in Example 2, except that compound 23 was used in place of compound 3.

### Example 12

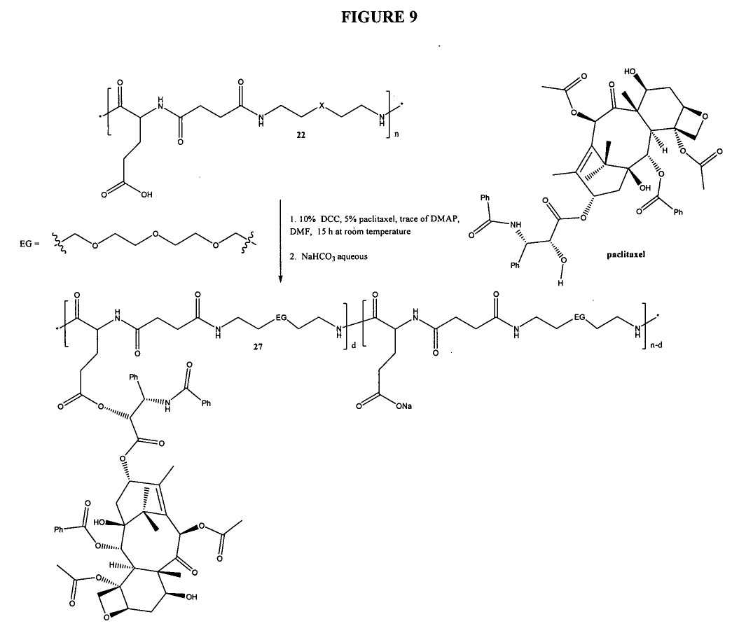

A polymer 26 was produced according to the reaction scheme illustrated in FIG. 8 as follows: Polymer 25 (57 mg) was dissolved in DMF (4 mL). Pac-NHS (50 mg) was added into the solution and continued to stir for 3 hours. The mixture was concentrated by rotary evaporation. The residue was partially redissolved in distilled water (5 mL). Polymer 26 was purified by Sephadex-G25 gel filtration. The product 26 was obtained in fractions after lyophilization.

### Example 13

A polymer 22 (105 mg) was dissolved in DMF (6 mL). 1,3-Dicyclohexylcarbodiimide (DCC, 0.1 equivalence per unit of 22) and paclitaxel (0.05 equivalence per unit of 22, LC Laboratories), and trace of amount of 1,4-dimethylaminopyridine (DMAP) were added into the solution. The mixture was stirred for 15 hours. The solvent was removed by rotary evaporation. A solution of sodium bicarbonate (10 mL, 1.0 M) was added into the residue. Precipitate was filtered through a 0.2 μm filer, and the crude polymer 27 was dialyzed in cellulose semipermeable membrane (cut-off 3,500 daltons) with distilled water for 12 hours at 4 degrees Celsius. The polymer 27 was obtained after lyophilization.

It will be understood by those of skill in the art that numerous and various modifications can be made without departing from the spirit of the present invention. Therefore, it should be clearly understood that the forms of the present invention are illustrative only and not intended to limit the scope of the present invention.

What is claimed is:

1. A polymer comprising a recurring unit of the formula (I): 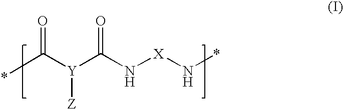
wherein:
X is selected from the group consisting of —(CH₂CH₂O)ₘ—CH₂CH₂— and —CH₂CH₂CH₂O—(CH₂CH₂O)ₘ—CH₂CH₂CH₂—, wherein m is an integer in the range of 1 to 100;
Y is selected from the group consisting of —C*HCH₂—, —C*HCH₂CH₂—, —C*H—NHC(═O)—CH₂CH₂—, —C*H—NHC(═O)—CH₂CH₂CH₂—, —CH₂CH₂N*CH₂CH₂—, —CH₂CH₂CH₂N*CH₂CH₂CH₂—, C₂ to C₂₀ alkyl, and C₆ to C₂₀ aryl, wherein C* and N* represent atoms to which Z is bonded; and
Z is selected from the group consisting of —NHR¹, —NH—C(═O)—(CH₂)ₙC(═O)NR¹R², —NH—C(═O)—(CH₂)ₙC(═O)OR¹, —(CH₂)ₙC(═O)NR¹R², —(CH₂)ₙC(═O)OR¹, —(CH₂)ₙC(═O)SR¹, and —(CH₂)ₙNR¹R², wherein n is an integer in the range of 1 to 3, wherein R¹ and R² are each independently selected from the group consisting of hydrogen, C₁ to C₂₀ alkyl, C₆ to C₂₀ aryl, anticancer drugs, peptides, antibody fragment, lactose, galactose, mannose, transferrin, magnetic resonance imaging agents, succinimyl, and alkali metal.

2. The polymer as recited in claim 1, wherein the formula (I) represents the following formula (II):

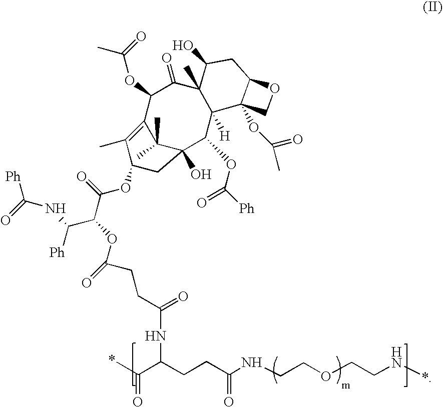

3. The polymer as recited in claim 1, wherein the formula (I) represents the following formula (III):

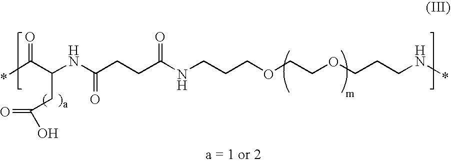

4. The polymer as recited in claim 1, wherein the formula (I) represents the following formula (IV):

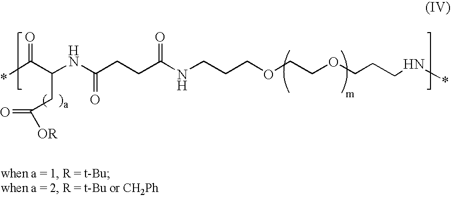

5. The polymer as recited in claim 1, wherein the formula (I) represents the following formula (V):

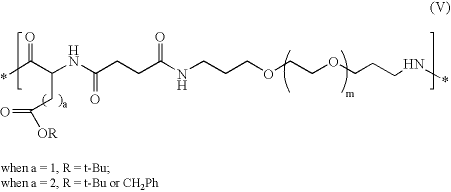

6. The polymer as recited in claim 1, wherein the formula (I) represents the following formula (VI):

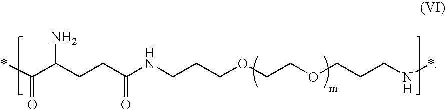

7. The polymer as recited in claim 1, wherein the formula (I) represents the following formula (VII):

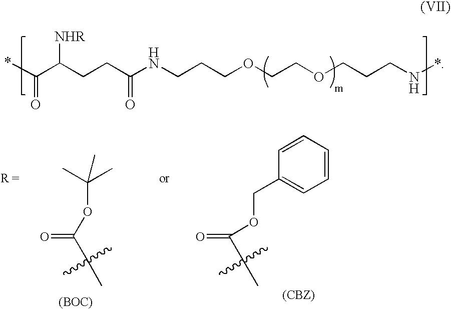

8. The polymer as recited in claim 1, wherein the formula (I) represents the following formula (VIII):

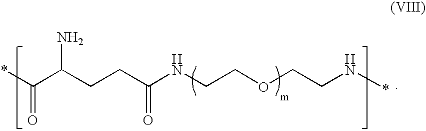

9. The polymer as recited in claim 1, wherein the formula (I) represents the following formula (IX):

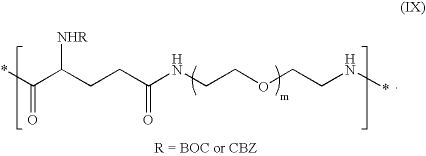

10. The polymer as recited in claim 1, which comprises at least one recurring unit selected from the group consisting of a recurring unit of the formula (II), a recurring unit of the formula (VIII), and a recurring unit of the formula (IX):

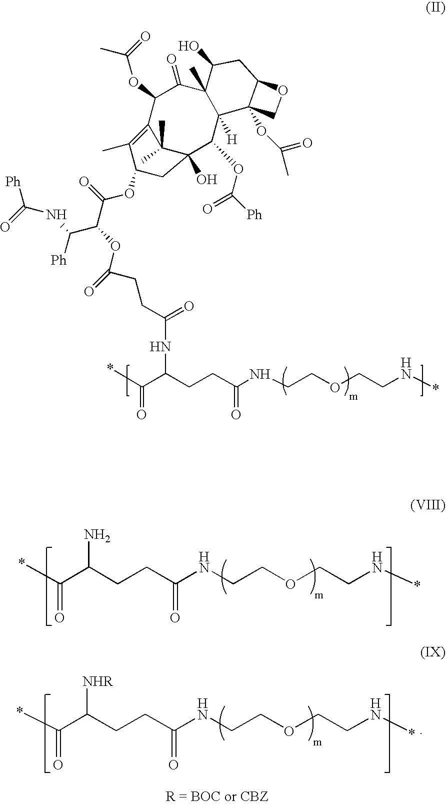

11. The polymer as recited in claim 10, which comprises a recurring unit of the formula (II), a recurring unit of the formula (VIII), and a recurring unit of the formula (IX).

12. A composition comprising a compound of the formula (X):

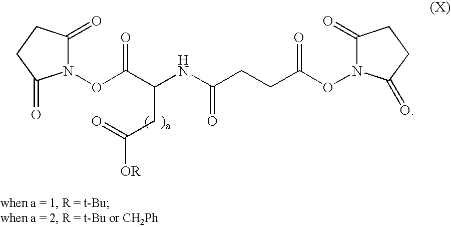

13. A method of producing the polymer of claim 2 comprising reacting a polymer comprising a recurring unit of the formula (VIII) with Pac-NHS:

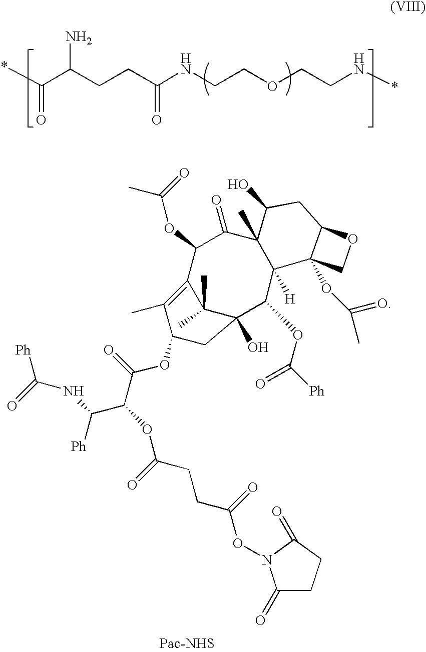

14. A method of producing the polymer of claim 3 comprising reacting a polymer comprising a recurring unit of the formula (IV) with trifluoacetic acid (TFA) or catalytic palladium/carbon hydrogenation:

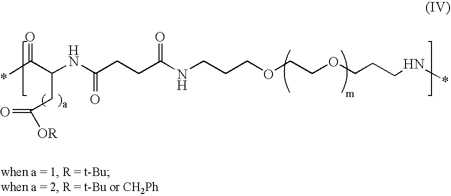

15. A method of producing the polymer of claim 4 comprising reacting a compound of the formula (X) with a compound of the formula (XII):

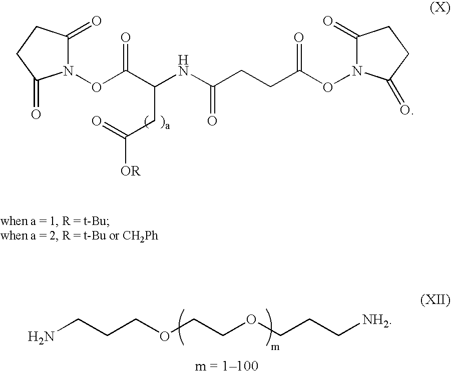

16. A method of producing the polymer of claim 5 comprising reacting a compound of the formula (X) with a compound of the formula (XIII):

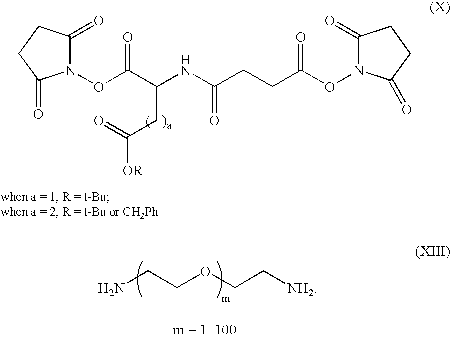

17. A method of producing the polymer of claim 6 comprising reacting a polymer comprising a recurring unit of the formula (VII) with trifluoroacetic acid (TFA) or catalytic palladium/carbon hydrogenation:

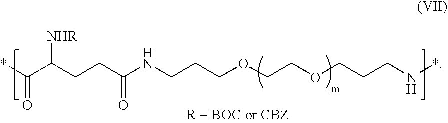

18. A method of producing the polymer of claim 7 comprising reacting a compound of the formula (XI) with a compound of the formula (XII):

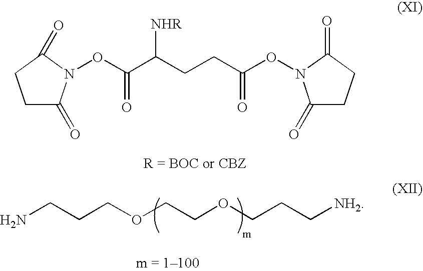

19. A method of producing the polymer of claim 8 comprising reacting a polymer comprising a recurring unit of the formula (IX) with trifluoroacetic acid (TFA) or catalytic palladium/carbon hydrogenation:

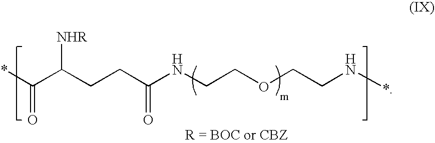

20. A method of producing the polymer of claim 9 comprising reacting a compound of the formula (XI) with a compound of the formula (XIII):

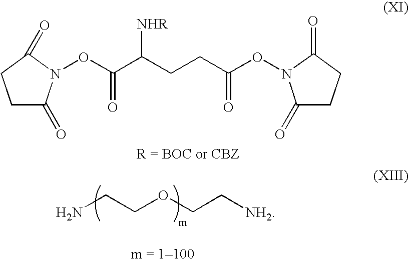

21. A method of producing the compound of claim 12 comprising reacting L-glutamic acid γ-benzyl ester, L-aspartic acid β-t-butyl ester, or L-glutamic acid γ-t-butyl ester with succinimide anhydride, followed by 1-(3-Dimethylaminopropyl)-3-ethylcardiimide hydrochloride (EDC), N-hydroxysuccinimide (NHS) coupling.
